(19) United States
(12) Patent Application Publication
(10) Pub. No.: US 2026/0256637 A1
(43) Pub. Date: Sep. 3, 2026

(54) SYSTEMS AND METHODS FOR MONITORING WEIGHT CHANGE TREND OF A PERSON IN BED

(71) Applicant: TOCHTECH TECHNOLOGIES LTD., Delta (CA)

(72) Inventors: Wei Fang, Langley (CA); Jing Yang, Richmond (CA)

(21) Appl. No.: 19/442,416

(22) Filed: Jan. 7, 2026

Related U.S. Application Data
(60) Provisional application No. 63/742,817, filed on Jan. 7, 2025.

(51) Int. Cl. A61G 7/05

(52) U.S. Cl. CPC A61G 7/0527

(57) ABSTRACT

A bed-sensor system monitors the weight-change trend of a person lying in a bed without requiring the person to stand on a scale. One or more weight sensors, such as load cells, are placed under support members of the bed, including at least two sensors at the head side of the bed. A processing module receives sensor signals and performs a calibration process to determine a calibration factor representing a proportion of the person's total weight borne by the head-side sensors. During use, the processing module monitors position-consistency parameters such as left-right weight distribution, determines weight values for the person at multiple time points using the calibration factor, and generates a weight-change trend based on the weight values. The system may perform auto-zero calibration during empty-bed periods, compute reliability metrics, and provide alerts or visualizations of weight-change trends to caregivers via wired or wireless user interfaces.

### CROSS-REFERENCE TO RELATED APPLICATIONS

This application claims the benefit of U.S. Provisional Patent Application No. 63/742,817 filed 7 Jan. 2025, the contents of which are hereby incorporated by reference.

### TECHNICAL FIELD

The present disclosure relates to systems and methods for health monitoring. In particular, the present disclosure relates to systems and methods for monitoring weight changes of a person in bed, without requiring the person to stand on a scale or otherwise be disturbed.

### BACKGROUND

Monitoring a person's body weight is an important component of health assessment and ongoing care, particularly in elderly patients, patients with chronic diseases (such as heart failure or renal disease), patients with limited mobility, and patients who are bedridden. Conventional methods typically require the patient to stand on a scale or otherwise physically transfer the patient onto a weighing platform. For many patients, this may be inconvenient, uncomfortable, and/or impractical and may require the assistance of caregivers.

Furthermore, such conventional methods for weighing patients typically involve weighing them intermittently, such as during clinic visits or at irregular times in the home. This makes it difficult to observe trends in the patients' weight change over time. In contrast, continuous or frequent monitoring of patients' weights is desirable because the rate of weight change, rather than the absolute weight values, often serves as a more sensitive indicator of clinical conditions, such as fluid retention, edema, dehydration, and/or malnutrition.

Existing bed scale systems may integrate weighing sensors into the bed frame or require the bed to be placed on a dedicated weighing platform. These systems can be expensive, may need to be tied to a specific bed, require lengthy manual operation, and/or can be impractical when the patient is moved to another bed or when the bed is repositioned. Environmental factors such as bedding or clothing variations may further introduce errors in absolute weight measurements and may limit the usefulness of such bed scale systems.

Accordingly, there is a need for a simple, low-cost, and non-invasive system for monitoring weight change trends in patients that can be used with existing beds, that can operate over long periods of time with minimal maintenance, and that can reliably determine the patients' weight change trends while being robust to variations in bedding, clothing, and/or patient posture.

### SUMMARY

In one aspect, a system for monitoring a weight change trend of a person lying in a bed comprises the bed, two or more weight sensors, and a processing module. The bed comprises and an upper frame and a plurality of support members. The upper frame comprises a head end and a foot end, with the upper frame adapted to receive the person. The plurality of support members extends from and supports the upper frame. At least two of the two or more weight sensors are disposed under one or more of the support members located proximate to the head end. The at least two of the two or more weight sensors comprises at least a first head-end sensor and a second head-end sensor. The processing module is operatively coupled to the two or more weight sensors. The processing module is configured to: (1) receive signals indicative of weight applied to the two or more weight sensors; (2) perform a calibration process to determine a calibration factor representing a proportion of a total weight of the person that is borne by the at least two of the two or more weight sensors; (3) monitor position-consistency parameters comprising a left-right weight distribution between the first head-end sensor and the second head-end sensor; (4) determine, over a plurality of time points, respective weight values associated with the person using the calibration factor and the signals from the at least two of the two or more weight sensors; and (5) generate a trend result value for the person over time based on the respective weight values.

In another aspect, the bed comprises four support members, wherein two of the four support members extend from the head end and two of the four support members extend from the foot end. The first head-end sensor and the second head-end sensor are disposed under the two of the four support members extending from the head end.

In yet another aspect, the processing module is configured to compute a left-right ratio between the signals from the first head-end sensor and the second head-end sensor during calibration and to compare a current left-right ratio to the calibrated left-right ratio to validate position consistency.

In still yet another aspect, the calibration process comprises: obtaining empty-bed readings from the first and second head-end sensors with the bed unoccupied; obtaining occupied-bed readings from the first and second head-end sensors with the person lying on the bed; and determining the calibration factor based at least in part on a difference between the occupied-bed readings and the empty-bed readings and on a known weight of the person.

In a further aspect, the processing module is further configured to perform an auto-zero calibration during detected empty-bed periods to compensate for sensor drift.

In still a further aspect, the processing module is further configured to apply one or more smoothing algorithms to the respective weight values to generate the trend result value.

In still yet a further aspect, the processing module is further configured to compute a reliability metric for the trend result value based on at least one of a number of readings from the first head-end sensor and the second head-end sensors, a stability of the position-consistency parameters, and a stability of the calibration factor.

In another aspect, the system further comprises a user device in communication with the processing module, the user device comprising a user interface configured to display the trend result value and to provide alerts when a magnitude of weight change exceeds a predetermined threshold.

In still another aspect, the processing module is configured to detect sudden changes in the signals indicative of at least one of bed movement, patient exit, or sensor malfunction, and to generate error notifications in response.

In a further aspect, the two or more weight sensors are implemented as load cells configured to be placed under the support members.

In still a further aspect, the processing module is further configured to store time-stamped weight values in a database and to compute daily or weekly weight-change rates based on the stored values.

In still yet a further aspect, the processing module is further configured to communicate the trend result value to a remote server via wireless communications interface.

In yet a further aspect, the processing module is further configured to ignore or down-weight signals that do not satisfy one or more position-consistency criteria.

In still yet a further aspect, the user interface comprises at least one of a mobile application, a web dashboard, or a nurse-station display.

In another aspect, a method for monitoring a weight-change trend of a person lying in a bed comprises: (1) placing a plurality of weight sensors under the bed comprising a head end and a foot end, the plurality of weight sensors comprising at least two of the plurality of weight sensors disposed under the bed proximate to the head end; (2) performing an initial calibration comprising: obtaining empty-bed readings from the at least two of the plurality of weight sensors disposed under the bed proximate to the head end; obtaining occupied-bed readings from the at least two of the plurality of weight sensors disposed under the bed proximate to the head end with the person lying on the bed; and computing a calibration factor based on a known weight of the person and on a difference between the occupied-bed readings and the empty-bed readings; (3) obtaining, at a plurality of time points, readings from the at least two of the plurality of weight sensors disposed under the bed proximate to the head end with the person lying on the bed; (4) evaluating a position consistency parameter using a left-right weight distribution between the at least two of the plurality of weight sensors disposed under the bed proximate to the head end; (5) determining, for readings that satisfy the position consistency parameter, respective weight values for the person based on the calibration factor; and (6) generating a trend result value for the person over time using the respective weight values.

In still another aspect, the method further comprises storing the respective weight values along with a time-stamp for each of the respective weight values, and further determining daily or weekly change in the trend result values.

In still yet another aspect, the method further comprises applying one or more smoothing algorithms to reduce noise in the respective weight values.

In a further aspect, the method further comprises generating an alert when the trend result value exceeds a predetermined threshold indicative of at least one of fluid retention, dehydration, or malnutrition.

In one aspect, the present disclosure provides a bed sensor system for monitoring the weight change trend of a person lying in a bed without disturbing the person. A plurality of weight sensors, such as load cells, are positioned under support points of the bed, for example under wheels or legs at the head end of the bed. The system uses the statically balanced nature of the bed-plus-patient system so that a relatively constant proportion of the total weight is borne by the sensors at the head side when the patient's position on the bed is substantially consistent.

Signals from the weight sensors are provided to a processing module configured to perform calibration, position-consistency monitoring, trending analysis, and error handling and compensation. The processing module determines a calibration factor that relates the total weight on the head-side sensors to the patient's total body weight, and monitors changes over time. Instead of requiring an exact absolute weight at each measurement, the system focuses on changes in the calibrated weight value to generate a weight-change metric or rate over a selected time interval.

In some aspects, the system records time-stamped weight readings, automatically detects periods when the bed is unoccupied to perform auto-zero calibration, monitors left/right weight distribution to confirm that the patient is in a comparable position, and computes moving averages and reliability metrics. The system may communicate the weight-change trend and associated alerts to user interfaces such as mobile applications, nurse-station dashboards, or cloud-based monitoring systems.

The disclosed systems and methods provide continuous or periodic monitoring of weight changes without requiring the patient to get out of bed or interact with a device, thereby improving comfort and supporting long-term monitoring with minimal caregiver workload.

The foregoing was intended as a summary only and of only some of the aspects. It was not intended to define the limits or requirements of the present disclosure. Other aspects will be appreciated by reference to the detailed description set out below.

### BRIEF DESCRIPTION OF THE DRAWINGS

The embodiments of the present disclosure will be described by reference to the drawings thereof, in which:

FIG. 1 depicts the system in accordance with one embodiment; and

FIG. 2 depicts a block diagram of the processing module.

### DETAILED DESCRIPTION

The embodiments described herein may be implemented in various forms. The description of specific embodiments is illustrative and not limiting-the scope of the invention is defined by the claims.

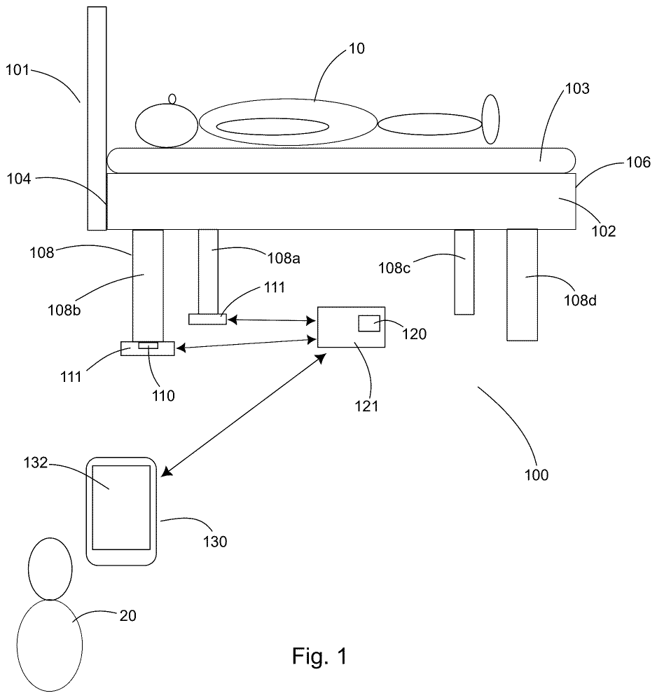

Referring to FIG. 1, in some embodiments, a system 100 for monitoring weight change in patients comprises a bed 101. The bed 101 comprises an upper frame 102 for supporting a mattress 103. The mattress 103 may be configured to support a patient 10. The upper frame 102 comprises a head end 104 and a foot end 106. The bed 101 further comprises a plurality of support members 108 adapted to support the upper frame 102. The support members 108 may take the form of bed legs or wheel assemblies, or a combination of both. In some embodiments, the bed 101 comprises four of the support members 108, such as, for example, support members 108a, 108b that are attached to the upper frame 102 proximate to the head end 104 and support members 108c, 108d that are attached to the upper frame 102 proximate to the foot end 106.

The system 100 further comprises a plurality of weight sensors 110 that are arranged under the bed 101. In some embodiments, the system 100 comprises two of the weight sensors 110, with one of each of the weight sensors 110 disposed under the support members 108 proximate to the head end 104 (e.g. support members 108a, 108b). In other embodiments, additional ones of the weight sensors 110 may also be disposed under the support members 108 proximate to the foot end 106 (e.g. support members 108c, 108d). For example, in some embodiments, the support members 108a, 108c may generally be disposed on the left side of the patient 10 when the patient 10 is lying on the bed 101, while the support members 108b, 108d may generally be disposed on the right side of the patient 10 when the patient 10 is lying on the bed 101.

The weight sensors 110 may be implemented as load cells, strain-gauge cells, force sensors, or other devices capable of converting mechanical load into electrical signals. In some embodiments, the weight sensors 110 may be located within a sensor housing 111 on which the support members 108 rest (as shown in FIG. 1). In other embodiments, the weight sensors 110 may be incorporated into the support members 108, such as into the wheel assemblies of the support members 108.

The weight sensors 110 may be coupled, via wireless or wired connections, to a processing module 120 that may be external to the weight sensors 110. The processing module 120 may be implemented using one or more processors, microcontrollers, and/or other processing circuitry. In some embodiments, the processing module 120 may be located under the bed 101, such as within a processor housing 121. In other embodiments, the processing module 120 may be integrated with other devices, such as integrated into a bedside monitoring device.

The processing module 120 may be configured to communicate with one or more user devices 130. The user devices 130 may include mobile devices (e.g. smartphones), tablets, computers, web dashboards, nurse station monitors, remote servers, or the like. Communications between the processing module 120 and the one or more user devices 130 may be through short-range or long-range wired or wireless connections, such as through Bluetooth, Wi-Fi, Ethernet, or cellular networks.

The patient 10 and the bed 101 on which the patient 10 lies may form a statically balanced system. Let the total weight of the bed 101 and the patient 10 be W. When the center of gravity of the combined patient 10 and bed 101 remains at a substantially constant position along the length of the bed 101, a generally constant proportion k of the total weight is borne by the support members 108a, 108b located proximate to the head end 104. Thus, a reading H from the weight sensors 110 disposed under the support members 108a, 108b may be represented by H=k×W.

When the weight of the patient 10 changes by ΔW while the position of the patient 10 remains sufficiently consistent, the resulting change in the readings from the weight sensors 110 located under the support members 108a, 108b may be approximately ΔH=k×ΔW. Accordingly, changes in the total weight ΔW may be inferred from changes in the readings from the weight sensors 110 disposed under the support members 108a, 108b. In some embodiments, it may not be necessary for the system 100 to determine the absolute value of W at each measurement; instead, it may be sufficient for the system 100 to track the calibrated readings from the weight sensors 110 disposed under the support members 108a, 108b to determine weight change trends.

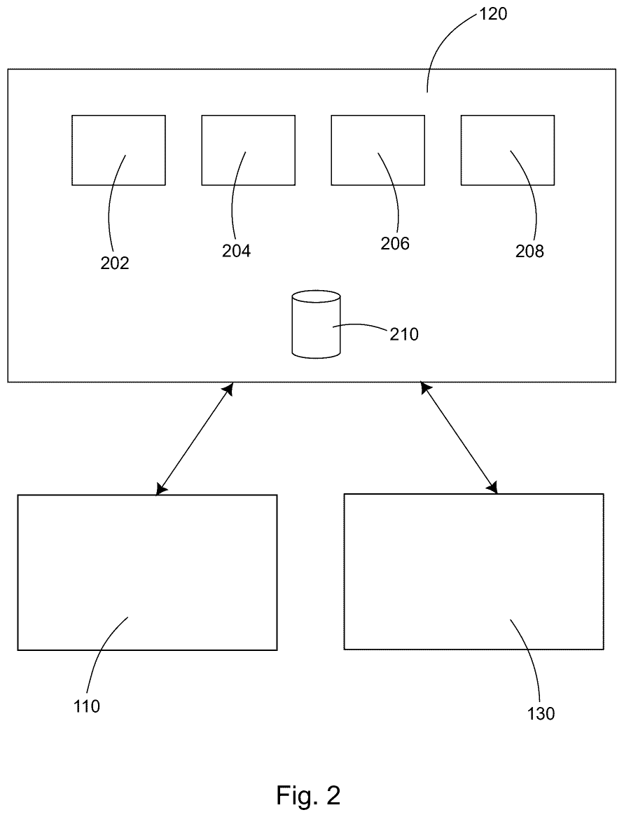

Referring to FIG. 2, the processing module 120 may be configured to receive readings from one or more of the weight sensors 110. The processing module 120 may comprise one or more of a calibration module 202, a position consistency module 204, a trending analysis module 206, and an error handling module 208. The processing module 120 may be configured to use or more of these modules based on the data received from the one or more of the weight sensors 110. These modules are discussed in more detail below.

In some embodiments, the calibration module 202 may be used to relate the readings from the weight sensors 110 disposed under the support members 108a, 108b (i.e. proximate to the head end 104) to the weight of the patient 10 and to compensate for the weight of the bed 101 and other offsets.

The calibration process is as follows. The calibration module 202 is first configured to determine an empty-bed measurement. When the bed 101 is unoccupied, the calibration module 202 is configured to receive readings from the weight sensors 110 disposed under the support members 108a, 108b (denoted as empty_left and empty_right, respectively). In some embodiments, if additional ones of the weight sensors 110 are present under one or both of the support members 108c, 108d, the calibration module 202 may be configured to receive readings from the weight sensors 110 disposed under the support members 108c, 108d. The sum of the empty-bed readings may be denoted as follows:
empty_head_sum=empty_left+empty_right.
   In embodiments where there are additional ones of the weight sensors 110 (e.g. present under one or both of the support members 108c, 108d), the constant proportion k may be equal to 1.

The calibration module 202 is then configured to determine an occupied-bed measurement. A known weight of the patient 10 is obtained using a conventional scale or other measurement technique. The known weight may be denoted as known_weight. When the patient 10 is lying on the bed 101 in a normal resting position, the calibration module 202 may be configured to receive readings from the weight sensors 110 disposed under the support members 108a, 108b (denoted as left_sensor and right_sensor, respectively). The sum of the occupied-bed readings may be denoted as follows:
head_sensors_total=left_sensor+right_sensor.

Based on the foregoing, the calibration module 202 may be configured to determine a left-right ratio as follows:
lr_ratio=left_sensor/right_sensor.
   This left-right ratio characterizes the lateral distribution of weight when the patient 10 is in a calibrated position. The calibration module 202 may also be configured to determine a proportionality factor k_factor as follows:
k_factor=(head_sensors_total−empty_head_sum)/known_weight
   In some embodiments, the use of the proportionality factor k_factor may be more accurate than the use of the constant proportion k, the proportionality factor k_factor minimizes or removes the effect of any center-of-gravity impacts.

The proportionality factor k_factor represents the proportion of the weight of the patient 10 reflected in the net head-end readings. In some embodiments, calibration data (e.g. k_factor, lr_ratio, empty_head_sum, etc.) may be stored by the processing module 120.

In some embodiments, the calibration module 202 may be configured to update the empty-bed readings and adjust offsets to compensate for drift. The calibration module 202 may carry this out during known empty-bed periods, such as when the weight sensors 110 detect zero or near-zero loads for longer than a predetermined threshold duration (e.g. for durations greater than 15 minutes). In addition, the calibration module 202 may also be configured to carry out recalibration routines periodically or upon detection of certain configuration changes (e.g. when the bed 101 is relocated).

The position consistency module 204 may be configured to ensure that weight-trend determinations are based on comparable patient positions. In some embodiments, the position consistency module 204 may be configured to receive readings from the weight sensors 110 disposed under the support members 108a, 108b (denoted as left_sensor_current and right_sensor_current, respectively) and to determine a current left-right ratio (denoted as lr_current) as follows:
lr_current=left_sensor_current/right_sensor_current

The position consistency module 204 may then be configured to compare the ratio lr_current with the calibrated ratio lr_ratio. If the difference between the ratios exceeds a predetermined threshold, the readings from the weight sensors 110 may be flagged as invalid or lower in reliability. The position consistency module 204 may also be configured to compare the total head-end load (plus optional foot-end load) against historical averages for the combination of the patient 10 and the bed 101. The position consistency module 204 may determine that sudden, large deviations indicate that the patient 10 is sitting up, lying on the edge of the bed 101, or away from the bed 101.

The position consistency module 204 may be configured to mark as valid any readings that satisfy predetermined criteria and to pass those readings to the trending analysis module 206. Furthermore, the readings that are marked as valid may also be time-stamped by the position consistency module 204. Readings that do not satisfy the predetermined criteria may be discarded by the position consistency module 204 or flagged to be down-weighted when used by the trending analysis module 206.

In some embodiments, with each valid reading, a net head-end weight component (denoted as H_net(t)) attributable to the patient 10 may be determined by the position consistency module 204 as follows:
H_net(t)=head_sensors_total(t)−empty_head_sum,
   where head_sensors_total(t) is the head_sensors_total at a particular time t, and the corresponding estimated weight of the patient 10 (denoted as W_est(t)) is determined as follows:
W_est(t)=H_net(t)/k_factor.

In some embodiments, the processing module 120 may not report W_est(t) directly to the user devices 130. Instead, the processing module 120 may use the values of W_est(t) internally to compute weight-change metrics, such as (1) daily or intra-day weight changes, determined by the following:
ΔW_day=W_est(t2)−W_est(t1),
   where t1 and t2 correspond to similar times on consecutive days; or (2) rate of change over a moving window, expressed as kg/day or percentage body weight change over a defined period.

In some embodiments, the trending analysis module 206 is configured to receive readings that have been considered valid and time-stamped by the position consistency module 204 (e.g. in the form of W_est(t) values).

Based on these readings, the trending analysis module 206 may be configured to store the time-stamped readings (e.g. in the form of W_est(t) values) in a time series database 210. The time series database 210 may be located in the processing module 120 or may be located external to the processing module 120. The W_est(t) values may be aggregated by the trending analysis module 206 into daily, weekly, and/or custom time intervals. In addition, the trending analysis module 206 may be configured to use moving-average, exponential smoothing, or other statistical filters to reduce noise in the data, which may be caused by small posture changes, sensor noise, and/or bedding variations.

In some embodiments, the trending analysis module 206 may be configured to determine gradual trends and/or short-term changes in the weight of the patient 10 (such as a steady weight gain over several days or sudden overnight increases) to determine an overall trend result value. In some embodiments, the trend result value may be expressed as a percentage change in weight over a specified interval of time (e.g. 3% increase in weight over the previous 3 months). The trend result value may be an indication of an overall degree of a rate of change of weight in the patient 10. Based on the data received by the trending analysis module 206, the trending analysis module 206 may be configured to assign a reliability metric to each computed trend result value, with the reliability metric based, at least in part, on the number of valid data points, the consistency of position metrics, and the stability of calibration parameters.

In some embodiments, the consistency of position metrics may be determined by the time of day, the left-right sensor ratio, the patient net weight, the k_factor, and/or the in-bed duration of the patient 10. The stability of calibration parameters may be determined by comparing current empty bed weight, patient net weight, left-right sensor ratio, etc. with initially calibrated values, as well as from the past multi-day (e.g. 7 days) validated data, to ensure that the current measured/determined values are within a reasonable range.

The trending analysis module 206 may be configured to transmit the trend result value(s) to the user devices 130. The user devices 130 may comprise a user interface 132 configured to display information to a user 20. For example, the user interface 132 may be configured to use the trend result value(s) received from the trending analysis module 206 to display a graph of weight change of the patient 10 over time. The user interface 132 may also be configured to highlight events where the weight change in the patient 10 exceeds predetermined thresholds. In some embodiments, the user interface 132 may also be configured to accept input from the user 20. For example, the user interface 132 may be configured to accept input from the user 20 relating to the predetermined thresholds for weight change (e.g. allowing the user 20 to set the predetermined thresholds).

The error handling module 208 may be configured to manage anomalous conditions and/or to maintain performance of the system 100. In some embodiments, the error handling module 208 may operate by itself or in conjunction with the other modules to conduct the following: (1) perform position consistency checks (as described above); (2) generate alerts for sudden changes in estimated weight that may indicate misplacement of the weight sensors 110, mechanical disturbance, and/or medical emergencies; (3) use statistical outlier detection to reject isolated readings inconsistent with neighboring data points; (4) execute regular system health checks (e.g. verifying sensor connectivity, battery status, self-test routines, etc.); and/or (5) apply sensor drift compensation based on repeated empty-bed measurements and long-term baseline monitoring.

The user interface 132 may be implemented as a mobile application, a web interface, a nurse station terminal display, and/or a cloud-based portal. In some embodiments, the user interface 132 may be configured to display current and historical weight trend graphs and/or to provide numerical indicators of daily or weekly weight change of the patient 10 and associated reliability metric(s). The user interface 132 may be configured to allow the user 20 to set and configure thresholds for alerts. For example, the user interface 132 may be configured by the user 20 to generate an alert if weight gain exceeds 2 kg in 3 days. The user device 130 may be configured to send alerts and/or other notifications to the user 20 through push messages, SMS messages, and/or email. In some embodiments, the user device 130 may also be configured to send alerts and/or other notifications to other clinical alarm systems.

In some embodiments, the processing module 120 may be configured to cause measurements from the weight sensors 110 to be taken every 5 minutes, although other intervals are also possible. In some embodiments, the processing module 120 may also be configured to use all of the measurements taken over a certain period of time (e.g. 24 hours) in order to determine an optimal measurement for that period of time. This determination may be based on an assessment conducted by the position consistency module 204. The optimal measurement may then be used by the trending analysis module 206 to determine the trend result value.

In some embodiments, the processing module 120 may be configured to communicate with remote servers for secure data storage, for analytics, for integration with electronic health record (EHR) systems, and/or for remote monitoring platforms.

In some embodiments, the system 100 may utilize two of the weight sensors 110, with each of the two weight sensors 110 disposed under the support members 108 proximate to the head end 104. However, in other embodiments, the system 100 may utilize additional ones of the weight sensors 110. For example, for a bed 101 comprising four support members 108, the system 100 may utilize four of the weight sensors 110, with each of the four weight sensors 110 disposed under each of the four support members 108. Regardless of the number of the weight sensors 110 used, similar principles of calibration and position consistency checking may still apply.

The system 100 may be used with different types of beds 101, such as hospital beds or beds in home care and institutional facilities, as long as the support members 108 allow for consistent contact with the weight sensors 110.

In some embodiments, the weight sensors 110 and the processing module 120 may be powered by mains electricity with battery backup. In other embodiments, the weight sensors 110 and the processing module 120 may be powered exclusively by batteries (e.g. rechargeable batteries). The system 100 may also employ power-saving modes when the patient 10 is determined to be not in the bed 101.

In some embodiments, the data relating to the weight change of the patient 10 may be encrypted by the processing module 120 for transmission and/or storage. Furthermore, access to the user interface 132 may be controlled through authentication mechanisms.

### EXAMPLE

For example, where the patient 10 has a known_weight of 60.1 kg, the following readings may be obtained from the weight sensors 110:
empty_left=15.8 kg
empty_right=17.4 kg
left_sensor=24.9 kg
right_sensor=24.3 kg

The calibration process may be determined as follows:
empty_head_sum=empty_left+empty_right=15.8 kg+17.4 kg=33.2 kg
head_sensors_total=left_sensor+right_sensor=24.9 kg+24.3 kg=49.2 kg
lr_ratio=left_sensor/right_sensor=24.9 kg/24.3 kg=1.025
k_factor=(head_sensors_total−empty_head_sum)/known_weight=(49.2 kg−33.2 kg)/60.1 kg=0.266

The following readings may be obtained from the weight sensors 110 after 2 months for the patient 10:
empty_left_current=15.7 kg
empty_right_current=17.3 kg
left_sensor_current=25.1 kg
right_sensor_current=24.4 kg

The processing module 120 may determine the weight of the patient 10 as follows:
empty_head_sum=empty_left_current+empty_right_current=15.7 kg+17.3 kg=33 kg
head_sensors_total=left_sensor_current+right_sensor_current=25.1 kg+24.4 kg =49.5 kg
lr_current=left_sensor_current/right_sensor_current=25.1 kg/24.4 kg=1.028
H_net(t)=head_sensors_total(t)−empty_head_sum=49.5 kg−33 kg=16.5 kg
W_est(t)=H_net(t)/k_factor=16.5 kg/0.266=62 kg

Based on the W_est(t) value of 62 kg, the processing module 120 may determine a trend result value of 3.16% increase over 2 months.

One of ordinary skill in the art will appreciate that the features described in connection with one embodiment may be combined with features of other embodiments unless stated otherwise. The scope of the invention is not limited to the precise structures and procedures described herein.

1. A system for monitoring a weight change trend of a person lying in a bed, the system comprising:
the bed comprising:
an upper frame comprising a head end and a foot end, the upper frame adapted to receive the person;
a plurality of support members extending from and supporting the upper frame;
two or more weight sensors, wherein at least two of the two or more weight sensors are disposed under one or more of the support members located proximate to the head end, and wherein the at least two of the two or more weight sensors comprises at least a first head-end sensor and a second head-end sensor; and
a processing module operatively coupled to the two or more weight sensors and configured to:
receive signals indicative of weight applied to the two or more weight sensors;
perform a calibration process to determine a calibration factor representing a proportion of a total weight of the person that is borne by the at least two of the two or more weight sensors;
monitor position-consistency parameters comprising a left-right weight distribution between the first head-end sensor and the second head-end sensor;
determine, over a plurality of time points, respective weight values associated with the person using the calibration factor and the signals from the at least two of the two or more weight sensors; and
generate a trend result value for the person over time based on the respective weight values.

2. The system of claim 1, wherein the bed comprises four support members, wherein two of the four support members extend from the head end and two of the four support members extend from the foot end, wherein the first head-end sensor and the second head-end sensor are disposed under the two of the four support members extending from the head end.

3. The system of claim 1, wherein the processing module is configured to compute a left-right ratio between the signals from the first head-end sensor and the second head-end sensor during calibration and to compare a current left-right ratio to the calibrated left-right ratio to validate position consistency.

4. The system of claim 1, wherein the calibration process comprises:
obtaining empty-bed readings from the first and second head-end sensors with the bed unoccupied;
obtaining occupied-bed readings from the first and second head-end sensors with the person lying on the bed; and
determining the calibration factor based at least in part on a difference between the occupied-bed readings and the empty-bed readings and on a known weight of the person.

5. The system of claim 1, wherein the processing module is further configured to perform an auto-zero calibration during detected empty-bed periods to compensate for sensor drift.

6. The system of claim 1, wherein the processing module is further configured to apply one or more smoothing algorithms to the respective weight values to generate the trend result value.

7. The system of claim 1, wherein the processing module is further configured to compute a reliability metric for the trend result value based on at least one of a number of readings from the first head-end sensor and the second head-end sensors, a stability of the position-consistency parameters, and a stability of the calibration factor.

8. The system of claim 1, further comprising a user device in communication with the processing module, the user device comprising a user interface configured to display the trend result value and to provide alerts when a magnitude of weight change exceeds a predetermined threshold.

9. The system of claim 1, wherein the processing module is configured to detect sudden changes in the signals indicative of at least one of bed movement, patient exit, or sensor malfunction, and to generate error notifications in response.

10. The system of claim 1, wherein the two or more weight sensors are implemented as load cells configured to be placed under the support members.

11. The system of claim 1, wherein the processing module is further configured to store time-stamped weight values in a database and to compute daily or weekly weight-change rates based on the stored values.

12. The system of claim 1, wherein the processing module is further configured to communicate the trend result value to a remote server via wireless communications interface.

13. The system of claim 1, wherein the processing module is further configured to ignore or down-weight signals that do not satisfy one or more position-consistency criteria.

14. The system of claim 8, wherein the user interface comprises at least one of a mobile application, a web dashboard, or a nurse-station display.

15. A method for monitoring a weight-change trend of a person lying in a bed, the method comprising:
placing a plurality of weight sensors under the bed comprising a head end and a foot end, the plurality of weight sensors comprising at least two of the plurality of weight sensors disposed under the bed proximate to the head end;
performing an initial calibration comprising:
obtaining empty-bed readings from the at least two of the plurality of weight sensors disposed under the bed proximate to the head end;
obtaining occupied-bed readings from the at least two of the plurality of weight sensors disposed under the bed proximate to the head end with the person lying on the bed; and
computing a calibration factor based on a known weight of the person and on a difference between the occupied-bed readings and the empty-bed readings;
obtaining, at a plurality of time points, readings from the at least two of the plurality of weight sensors disposed under the bed proximate to the head end with the person lying on the bed;
evaluating a position consistency parameter using a left-right weight distribution between the at least two of the plurality of weight sensors disposed under the bed proximate to the head end;
determining, for readings that satisfy the position consistency parameter, respective weight values for the person based on the calibration factor; and
generating a trend result value for the person over time using the respective weight values.

16. The method of claim 15, further comprising storing the respective weight values along with a time-stamp for each of the respective weight values, and further determining daily or weekly change in the trend result values.

17. The method of claim 15, further comprising applying one or more smoothing algorithms to reduce noise in the respective weight values.

18. The method of claim 15, further comprising generating an alert when the trend result value exceeds a predetermined threshold indicative of at least one of fluid retention, dehydration, or malnutrition.

19. A non-transitory computer-readable medium storing instructions which, when executed by one or more processors, cause the processors to perform operations comprising the steps of the method of claim 15.
